(19) United States
(12) Patent Application Publication
(10) Pub. No.: US 2006/0078615 A1
(43) Pub. Date: Apr. 13, 2006

(54) Bilayer tablet of telmisartan and simvastatin

(75) Inventor: Anja Kohlrausch, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/236,911

(22) Filed: Sep. 28, 2005

(30) Foreign Application Priority Data
Oct. 12, 2004 (EP) 04 024 239.8

(51) Int. Cl. A61K 9/24

(52) U.S. Cl. 424/471

(57) ABSTRACT

A bilayer tablet comprises a first layer formulated for instant release of the angiotensin II receptor antagonist telmisartan from a dissolving tablet matrix and a second layer formulated for instant release of the HMG-CoA reductase inhibitor simvastatin from a disintegrating or eroding tablet matrix.

### RELATED APPLICATIONS

This application claims priority to European Application No. EP 04 024 239.8, filed Oct. 12, 2004, which is hereby incorporated by reference in its entirety.

### FIELD OF THE INVENTION

The present invention relates to a pharmaceutical tablet comprising a first layer of the angiotensin II receptor antagonist telmisartan in a dissolving tablet matrix and a second layer of the HMG-CoA reductase inhibitor simvastatin in a disintegrating or eroding tablet matrix.

### BACKGROUND OF THE INVENTION

Telmisartan is an angiotensin II receptor antagonist developed for the treatment of hypertension and other medical indications as disclosed in EP-A-502314. Its chemical name is 4′-[2-n-propyl-4-methyl-6-(1-methylbenzimidazol-2-yl)benzimidazol-1-ylmethyl]biphenyl-2-carboxylic acid having the following structure:

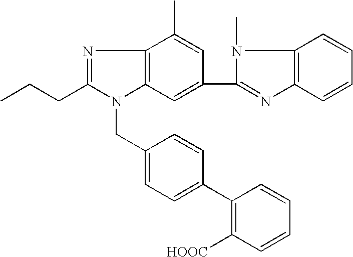

Telmisartan is manufactured and supplied in the free acid form. It is characterized by its very poor solubility in aqueous systems at the physiological pH range of the gastrointestinal tract of between pH 1 to 7. As disclosed in WO 00/43370, crystalline telmisartan exists in two polymorphic forms having different melting points. Under the influence of heat and humidity, the lower melting polymorph B transforms irreversibly into the higher melting polymorph A.

Simvastatin disclosed in EP-A-033538 is a long-acting HMG-CoA reductase inhibitor with the chemical name (1S,3R,7S,8S,8aR)-8-[2-[(2R,4R)-4-hydroxy-6-oxotetrahydro-2H-pyran-2-yl]ethyl]-3,7-dimethyl-1,2,3,7,8,8a-hexahydronaphtahlene-1-yl-2,2-dimethylbutanoate or alternatively (βR,δR,1S)-8β-(2,2-dimethylbutyryloxy)-1,2,6,7,8,8aα-hexahydro-β,δ-dihydroxy-2α,6β-dimethyl-1α-naphthaleneheptanoic acid δ-lactone having the following structure:

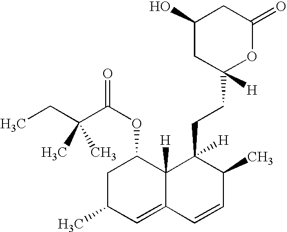

“Statins” are a class of drugs that lower the level of cholesterol in the blood by reducing the production of cholesterol by the liver. Statins block the enzyme in the liver that is responsible for making cholesterol. This enzyme is called 3-hydroxy-3-methylglutaryl-coenzyme-A reductase or β-hydroxy-β-methylglutaryl-coenzyme-A reductase (HMG-CoA reductase). Scientifically, statins are called HMG-CoA reductase inhibitors.

Statins are used for preventing and treating atherosclerosis that causes chest pain, heart attacks, strokes, and intermittent claudication in individuals who have or are at risk for atherosclerosis. Risk factors for atherosclerosis include abnormally elevated cholesterol levels, a family history of heart attacks (particularly at a young age), increasing age, and diabetes. Most individuals are placed on statins because of high levels of cholesterol.

### OBJECT OF THE INVENTION

The mechanisms of action of telmisartan and simvastatin are considered to cooperate favorably in the treatment or prevention of a condition selected from the group consisting of stroke, myocardial infarction, transient ischemic attack, congestive heart failure, cardiovascular disease, diabetes, insulin resistance, impaired glucose tolerance, pre-diabetes, type 2 diabetes mellitus, metabolic syndrome (syndrome X), obesity, hypertriglyceridemia, elevated serum concentration of C-reactive protein, elevated serum concentration of lipoprotein(a), elevated serum concentration of homocysteine, elevated serum concentration of low-density lipoprotein (LDL)-cholesterol, elevated serum concentration of lipoprotein-associated phospholipase (A2), reduced serum concentration of high density lipoprotein (HDL)-cholesterol, reduced serum concentration of HDL(2b)-cholesterol, reduced serum concentration of adiponectin, cognitive decline, and dementia, either alone or in combination with the treatment of hypertension.

As this conclusion gets supported by an increasing amount of clinical data, there is an increasing desire for a fixed dose combination drug comprising the active ingredients telmisartan and simvastatin. However, both telmisartan and simvastatin are chemical compounds difficult to handle. Therefore, an oral fixed dose combination drug which combines the features of pharmacologic efficacy, adequate drug stability and a reliable and robust method of manufacture has to overcome a number of technical problems. It is an object of the present invention to provide such a fixed dose combination drug. There are various types of fixed dose dosage forms conceivable but it cannot be predicted which of these dosage forms best combines product stability, pharmacological efficacy, and reliable manufacture. Examples of such dosage forms are oral osmotic systems (OROS), coated tablets, matrix tablet, bilayer tablets, and the like. The present invention is based on the recognition, that the dosage form, which best combines adequate drug stability, optimum drug release of both active ingredients, pharmacological efficacy, and reliable manufacture for a combination of telmisartan and simvastatin, is a bilayer tablet. Generally, a fixed-dose combination of drugs intended for instant release is prepared by either making a powder mixture or a co-granulate of the two active ingredients with the necessary excipients, normally keeping the basic formulation of the corresponding mono-drug preparation and simply adding the second drug component.

With a combination of telmisartan and simvastatin, this approach does not appear feasible due to the incompatibility of simvastatin with components of the conventional telmisartan formulations.

Another approach is to produce separate film-coated tablets for telmisartan and simvastatin in such a size and shape that these can be filled into a capsule. Large capsules would be required for the high dose combinations, which is not preferable with regard to patients' compliance.

### SUMMARY OF THE INVENTION

In accordance with the present invention, problems associated with the preparation of a fixed dose combination drug comprising telmisartan and simvastatin can best be handled by means of a bilayer pharmaceutical tablet comprising a first layer of telmisartan, preferably in substantially amorphous form, in a dissolving tablet matrix and a second layer of simvastatin in a disintegrating or eroding tablet matrix.

The tablet according to the present invention provides a largely pH-independent dissolution of the poorly water-soluble telmisartan, thereby facilitating dissolution of the drug at a physiological pH level, and adequate stability and drug release of simvastatin. The tablet structure also overcomes the stability problem caused by the incompatibility of Simvastatin with basic constituents of telmisartan.

### DEFINITIONS

As used herein, the term “substantially amorphous” refers to a product comprising amorphous constituents in a proportion of at least 90%, preferably at least 95%, as determined by X-ray powder diffraction measurement.

The term “dissolving tablet matrix” refers to a pharmaceutical tablet base formulation having instant release (fast dissolution) characteristics that readily dissolves in a physiological aqueous medium.

The term “disintegrating or eroding tablet matrix” refers to a pharmaceutical tablet base formulation having instant release characteristics that readily disintegrates or erodes in a physiological aqueous medium.

### DESCRIPTION OF THE INVENTION

A fixed dose combination according to the present invention represents a pharmaceutical bilayer tablet comprising a first layer of telmisartan in substantially amorphous form and a second layer of simvastatin in a disintegrating or eroding tablet matrix.

The active ingredient telmisartan is generally supplied in its free acid form, although pharmaceutically acceptable salts such as the sodium salt may also be used. Since during subsequent processing telmisartan is normally dissolved and transformed into a substantially amorphous form, its initial crystal morphology and particle size are of little importance for the physical and biopharmaceutical properties of the bilayer tablet formulation obtained. It is, however, preferred to remove agglomerates from the starting material, e.g., by sieving, in order to facilitate wetting and dissolution during further processing.

Substantially amorphous telmisartan may be produced by any suitable method known to those skilled in the art, for instance, by freeze-drying of aqueous solutions, coating of carrier particles in a fluidized bed, and solvent deposition on sugar pellets or other carriers. Preferably, however, the substantially amorphous telmisartan is prepared by the specific spray-drying method described in WO 03/059327 (corresponding to U.S. patent Application Pub. No. 2005/0089575, which is hereby incorporated by reference). A bilayer tablet according to the present invention generally contains 10 mg to 160 mg, preferably 20 mg to 80 mg or 40 mg to 80 mg, of telmisartan; and 1 mg to 100 mg, preferably 5 mg to 80 mg, of simvastatin. Preferred dose strengths of telmisartan are 20 mg, 40 mg, and 80 mg; preferred dose strengths of simvastatin are 5 mg, 10 mg, 20 mg, 40 mg, and 80 mg. Presently preferred forms are bilayer tablets comprising 20/80 mg, 40/80 mg, 80/80 mg, 20/40 mg, 40/40 mg, 80/40 mg, 20/20 mg, 40/20 mg, 80/20 mg, 20/10 mg, 40/10 mg, 80/10 mg, 20/5 mg, 40/5 mg, and 80/5 mg, of telmisartan and simvastatin, respectively.

The first tablet layer contains telmisartan in substantially amorphous form dispersed in a dissolving tablet matrix having instant release (fast dissolution) characteristics. The dissolving tablet matrix may have neutral or basic properties, although a basic tablet matrix is preferred.

In such a preferred embodiment, the dissolving matrix of the telmisartan layer comprises a basic agent, a water-soluble diluent and, optionally, other excipients and adjuvants. Specific examples of suitable basic agents are alkali metal hydroxides such as NaOH and KOH; basic amino acids such as arginine and lysine; and meglumine (N-methyl-D-glucamine), NaOH and meglumine being preferred.

Specific examples of suitable water-soluble diluents are carbohydrates such as monosaccharides like glucose; oligosaccharides like sucrose, anhydrous lactose and lactose monohydrate; and sugar alcohols like sorbitol, mannitol, erythrol, and xylitol. Sorbitol is a preferred diluent.

The other excipients and/or adjuvants are, for instance, selected from binders, carriers, fillers, lubricants, flow control agents, crystallization retarders, solubilizers, coloring agents, pH control agents, surfactants, and emulsifiers, specific examples of which are given below in connection with the second tablet layer composition. The excipients and/or adjuvants for the first tablet layer composition are preferably chosen such that a non-acidic, fast dissolving tablet matrix is obtained.

The first tablet layer composition generally comprises 3 to 50 wt. %, preferably 5 to 35 wt. %, of active ingredient; 0.25 to 20 wt. %, preferably 0.40 to 15 wt. %, of basic agent; and 30 to 95 wt. %, preferably 60 to 80 wt. % of water-soluble diluent (filler). Other (optional) constituents may, for instance, be chosen from one or more of the following excipients and/or adjuvants in the amounts indicated:
10 to 30 wt. %, preferably 15 to 25 wt. %, of binders, carriers and fillers, thereby replacing the water-soluble diluent;
0.1 to 5 wt. %, preferably 0.5 to 3 wt. %, of lubricants;
0.1 to 5 wt. %, preferably 0.3 to 2 wt. %, of flow control agents;
1 to 10 wt. %, preferably 2 to 8 wt. %, of crystallization retarders;
1 to 10 wt. %, preferably 2 to 8 wt. %, of solubilizers;
0.05 to 1.5 wt. %, preferably 0.1 to 0.8 wt. %, of coloring agents;
0.5 to 10 wt. %, preferably 2 to 8 wt. %, of pH control agents; and
0.01 to 5 wt. %, preferably 0.05 to 1 wt. %, of surfactants and emulsifiers.

The second tablet layer composition comprises simvastatin dispersed in a disintegrating or eroding tablet matrix having instant release (fast dissolution) characteristics. The disintegrating or eroding tablet matrix may have weakly acidic, neutral, or weakly basic properties, a neutral tablet matrix being preferred.

In a preferred embodiment, the disintegrating or eroding matrix comprises one or more fillers, a lubricant, an antioxidant and, optionally a binder or polymer, a disintegrant, other excipients and adjuvants.

Preferred fillers for the second layer are selected from the group consisting of pregelatinized starch, microcrystalline cellulose, cellulose, mannitol, erythritol, lactose monohydrate, calcium hydrogenphosphate, sorbitol, and xylitol. Particularly preferred are pregelatinized starch, microcrystalline cellulose and lactose monohydrate.

Preferred lubricants are sodium stearyl fumarate and magnesium stearate. Particularly preferred is magnesium stearate.

Preferred antioxidants are butylated hydroxyanisole, ascorbic acid, ascorbyl palmitate, butylated hydroxytoluene and sodium metabisulfite. Particularly preferred is butylated hydroxyanisole.

Preferred disintegrants are selected from the group consisting of croscarmellose sodium salt (cellulose carboxymethylether sodium salt, crosslinked), sodium starch glycolate, crosslinked polyvinylpyrrolidone (crospovidone), corn starch, and low-substituted hydroxypropylcellulose. Particularly preferred are sodium starch glycolate and croscarmellose sodium salt.

Preferred binders are selected from the group consisting of polyvinyl pyrrolidone (Povidone), copolymers of vinylpyrrolidone with other vinylderivatives (Copovidone), hydroxypropylmethylcellulose, methylcellulose, and hydroxypropylcellulose. Particularly preferred are hydroxypropylmethylcellulose and copovidone.

The second tablet layer composition generally comprises 1 to 80 wt. %, preferably 5 to 40 wt. % of simvastatin and 10 to 99 wt. %, preferably 25 to 95 wt. % of fillers.

The other excipients and/or adjuvants are, for instance, selected from binders (0 to 7 wt. %, preferably 1 to 4 wt. %), disintegrants (0 to 10 wt. %, preferably 1 to 4 wt. %), lubricants (0.25 to 3 wt. %, preferably 0.5 to 2 wt. %), antioxidants, chelating agents, and coloring agents, specific examples of which are also given below. The excipients and/or adjuvants for the second tablet layer composition are preferably chosen such that a neutral, disintegrating or eroding tablet matrix is obtained.

As solvent for the granulation liquid, which, as a volatile component, does not remain in the final product, methanol, ethanol, isopropanol, or purified water can be used; preferred solvents are ethanol and purified water.

The other excipients and adjuvants, if used, are coloring agents including dyes and pigments such as iron oxides. Examples for chelating agents are citric acid and sodium citrate.

The layers can be differentiated by using different colors.

For preparing a bilayer tablet according to the present invention, the first and second tablet layer compositions may be compressed in the usual manner in a bilayer tablet press, e.g., a high-speed rotary press in a bilayer tabletting mode. However, care should be taken not to employ an excessive compression force for the first tablet layer. Preferably, the ratio of the compression force applied during compression of the first tablet layer to the compression force applied during compression of both the first and second tablet layers is in the range of from 1:10 to 1:2. For instance, the first tablet layer may be compressed at moderate force of 4 to 8 kN, whereas the main compression of first plus second layer is performed at a force of 10 to 20 kN.

During bilayer tablet compression adequate bond formation between the two layers is achieved by virtue of distance attraction forces (intermolecular forces) and mechanical interlocking between the particles.

The bilayer tablets obtained release the active ingredients rapidly and in a largely pH-independent fashion, with complete release occurring within less than 60 minutes and release of the major fraction occurring within less than 15 minutes.

In accordance with the present invention, a substantially increased dissolution rate of the active ingredients and, in particular, of telmisartan is achieved. Normally, at least 70% and typically at least 90% of the drug load are dissolved after 30 minutes.

The bilayer tablets of the present invention tend to be slightly hygroscopic and are therefore preferably packaged using a moisture-proof packaging material such as aluminum foil blister packs, or polypropylene tubes and HDPE bottles which preferably contain a desiccant.

A preferred method of producing the bilayer tablet according to the present invention comprises
(i) providing a first tablet layer composition by:
   a) preparing an aqueous solution of telmisartan, at least one basic agent and, optionally, a solubilizer and/or a crystallization retarder;
   b) spray-drying said aqueous solution to obtain a spray-dried granulate;
   c) mixing said spray-dried granulate with a water-soluble diluent to obtain a premix;
   d) mixing said premix with a lubricant to obtain a final blend for the first layer; and
   e) optionally, adding other excipients and/or adjuvants in any of steps a) to d);
(ii) providing a second tablet layer composition comprising simvastatin;
(iii) compressing each of the first and second tablet layer composition to form a tablet layer; and
(iv) compressing the separate tablet layers to form a bilayer tablet.

To provide a first tablet layer composition, an aqueous alkaline solution of telmisartan is prepared by dissolving the active ingredient in purified water with the help of one or more basic agents like sodium hydroxide and meglumine. Optionally, a solubilizer and/or a recrystallization retarder may be added. The dry matter content of the starting aqueous solution is generally 10 to 40 wt. %, preferably 20 to 30 wt. %. The aqueous solution is then spray-dried at room temperature or preferably at increased temperatures of, for instance, between 50° C. and 100° C. in a co-current or countercurrent spray-drier at a spray pressure of, for instance, 1 to 4 bar. Generally speaking, the spray-drying conditions are preferably chosen in such a manner that a spray-dried granulate having a residual humidity of <5 wt. %, preferably <3.5 wt. %, is obtained in the separation cyclone. To that end, the outlet air temperature of the spray-drier is preferably kept at a value of between about 80° C. and 90° C. while the other process parameters such as spray pressure, spraying rate, inlet air temperature, etc., are adjusted accordingly.

The spray-dried granulate obtained is preferably a fine powder having the following particle size distribution:
d₁₀: ≦20 μm, preferably ≦10 μm
d₅₀: ≦80 μm, preferably 20 to 55 μm
d₉₀: ≦350 μm, preferably 50 to 150 μm

After spray-drying, the active ingredient telmisartan as well as the excipients contained in the spray-dried granulate are in a substantially amorphous state with no crystallinity being detectable. From a physical point of view, the spray-dried granulate is a solidified solution or glass having a glass transition temperature T_{g} of preferably >50° C., more preferably >80° C.

Based on 100 parts by weight of active ingredient telmisartan, the spray-dried granulate preferably contains 5 to 200 parts by weight of basic agent and, optionally, solubilizer and/or crystallization retarder.

The water-soluble diluent is generally employed in an amount of 30 to 95 wt. %, preferably 60 to 80 wt. %, based on the weight of the first tablet layer composition.

The lubricant is generally added to the premix in an amount of 0.1 to 5 wt. %, preferably 0.3 to 2 wt. %, based on the weight of the first tablet layer composition.

Mixing is carried out in two stages, i.e., in a first mixing step the spray-dried granulate and the diluent are admixed using, e.g., a high-shear mixer or a free-fall blender, and in a second mixing step the lubricant is blended with the premix, preferably also under conditions of high shear. The method of the invention is however not limited to these mixing procedures and, generally, alternative mixing procedures may be employed in steps c), d), and also in the subsequent steps f) and g), such as, e.g., container mixing with intermediate screening.

To provide a second tablet layer composition comprising simvastatin, simvastatin and part of the excipients (for example, lactose monohydrate, microcrystalline cellulose, pregelatinized starch, stabilizing agents) are premixed and granulated with the granulation liquid using a high shear granulator. The granulation liquid contains a solvent (for example, purified water or ethanol) and optional stabilizing agents (for example, antioxidants like ascorbic acid and butylated hydroxyanisole) and optional a binder. After high shear granulation, the granulate is wet sieved through an appropriate sieve and subsequently dried using a fluid bed granulator or a vacuum tray dryer. The dried granules are sieved through an appropriate sieve. After addition of the lubricant (for example, magnesium stearate) and optional disintegrants (for example, sodium starch glycolate), the mixture is blended in a free fall blender. Alternative methods for granulation of active ingredient and excipients with the granulation liquid are fluid bed granulation or one pot granulation.

First and second tablet layer compositions as described above can be compressed into bilayer tablets of the target tablet weight with appropriate size and crushing strength, using an appropriate tablet press. Optional an appropriate external lubricant spray system for the dies and punches can be used during manufacturing of tablets in order to improve lubrication.

For the production of bilayer tablets according to the present invention, the separate tablet layer compositions can be compressed in a bilayer tablet press, e.g., a rotary press in the bilayer tabletting mode, in the manner described above. In order to avoid any cross-contamination between the tablet layers (which could lead to decomposition of simvastatin), any granulate residues have to be carefully removed during tabletting by intense suction of the die table within the tabletting chamber.

In order to further illustrate the present invention, the following non-limiting examples are given.

### FORMULATION EXAMPLES

### Example 1

### Telmisartan 80 mg/Simvastatin 80 mg 2-Layer Tablets

| | mg | % of Telmisartan- | % of Simvastatin- |
|---|---|---|---|
| Constituents | per tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Sorbitol | 337.280 | 70.267 | |
| Magnesium stearate | 8.000 | 1.667 | |
| Purified water* | * | * | |
| Total | 480.000 | 100.000 | |
| Telmisartan-layer | | | |
| Simvastatin | 80.000 | | 40.000 |
| Microcrystalline | 20.000 | | 10.000 |
| cellulose | | | |
| Lactose monohydrate | 73.480 | | 36.740 |
| Pregelatinized starch | 20.000 | | 10.000 |
| Butylated | 0.020 | | 0.010 |
| hydroxyanisole | | | |
| Ascorbic acid | 5.000 | | 2.500 |
| Magnesium stearate | 1.500 | | 0.750 |
| Purified water* | * | | * |
| Ethanol* | * | | * |
| Total | 200.000 | | 100.000 |
| Simvastatin-layer | | | |
| Total 2-layer tablet | 680.000 | | |

*Volatile component, does not remain in final product

### Example 2

### Telmisartan 80 mg/Simvastatin 80 mg 2-Layer Tablets

| | mg per | % of Telmisartan- | % of Simvastatin- |
|---|---|---|---|
| Constituents | tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Sorbitol | 337.280 | 70.267 | |
| Magnesium stearate | 8.000 | 1.667 | |
| Purified water* | * | * | |
| Total Telmisartan-layer | 480.000 | 100.000 | |
| Simvastatin | 80.000 | | 40.000 |
| Microcrystalline | 40.000 | | 20.000 |
| cellulose | | | |
| Lactose monohydrate | 68.460 | | 34.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 680.000 | | |

*Volatile component, does not remain in final product

### Example 3

### Telmisartan 80 mg/Simvastatin 20 mg 2-Layer Tablets

| | | | % |
|---|---|---|---|
| | mg | % of Telmisartan- | of Simvastatin- |
| Constituents | per tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Sorbitol | 337.280 | 70.267 | |
| Magnesium stearate | 8.000 | 1.667 | |
| Purified water* | * | * | |
| Total Telmisartan-layer | 480.000 | 100.000 | |
| Simvastatin | 20.000 | | 10.000 |
| Microcrystalline | 20.000 | | 10.000 |
| cellulose | | | |
| Lactose monohydrate | 132.980 | | 66.490 |
| Pregelatinized starch | 20.000 | | 10.000 |
| Butylated hydroxyanisole | 0.020 | | 0.010 |
| Ascorbic acid | 5.000 | | 2.500 |
| Magnesium stearate | 2.000 | | 1.000 |
| Purified water* | * | | * |
| Ethanol* | * | | * |
| Total Simvastatin-layer | 200.000 | | 100.000 |
| Total 2-layer tablet | 680.000 | | |

*Volatile component, does not remain in final product

### Example 4

### Telmisartan 20 mg/Simvastatin 5 mg 2-Layer Tablets

| | mg per | % of Telmisartan- | % of Simvastatin- |
|---|---|---|---|
| Constituents | tablet | layer | layer |
| Telmisartan | 20.000 | 16.667 | |
| Sodium hydroxide | 1.680 | 1.400 | |
| Povidone | 6.000 | 5.000 | |
| Meglumine | 6.000 | 5.000 | |
| Sorbitol | 84.320 | 70.267 | |
| Magnesium stearate | 2.000 | 1.667 | |
| Purified water* | * | * | |
| Total Telmisartan-layer | 120.000 | 100.000 | |
| Simvastatin | 5.000 | | 2.500 |
| Microcrystalline | 20.000 | | 10.000 |
| cellulose | | | |
| Lactose monohydrate | 147.980 | | 73.990 |
| Pregelatinized starch | 20.000 | | 10.000 |
| Butylated hydroxyanisole | 0.020 | | 0.010 |
| Ascorbic acid | 5.000 | | 2.500 |
| Magnesium stearate | 2.000 | | 1.000 |
| Purified water* | * | | * |
| Ethanol* | * | | * |
| Total Simvastatin-layer | 200.000 | | 100.000 |
| Total 2-layer tablet | 320.000 | | |

*Volatile component, does not remain in final product

### Example 5

### Telmisartan 40 mg/Simvastatin 40 mg 2-Layer Tablets

| | mg per | % of Telmisartan- | % of Simvastatin- |
|---|---|---|---|
| Constituents | tablet | layer | layer |
| Telmisartan | 40.000 | 16.667 | |
| Sodium hydroxide | 3.360 | 1.400 | |
| Povidone | 12.000 | 5.000 | |
| Meglumine | 12.000 | 5.000 | |
| Sorbitol | 168.640 | 70.267 | |
| Purified water* | 4.000 | 1.667 | |
| Magnesium stearate | * | * | |
| Total Telmisartan-layer | 240.000 | 100.000 | |
| Simvastatin | 40.000 | | 20.000 |
| Microcrystalline | 20.000 | | 10.000 |
| cellulose | | | |
| Lactose monohydrate | 112.980 | | 56.490 |
| Pregelatinized starch | 20.000 | | 10.000 |
| Butylated hydroxyanisole | 0.020 | | 0.010 |
| Ascorbic acid | 5.000 | | 2.500 |
| Magnesium stearate | 2.000 | | 1.000 |
| Purified water* | * | | * |
| Ethanol* | * | | * |
| Total Simvastatin-layer | 200.000 | | 100.000 |
| Total 2-layer tablet | 440.000 | | |

*Volatile component, does not remain in final product

### Example 6

### Telmisartan 40 mg/Simvastatin 80 mg 2-Layer Tablets

| | mg per | % of Telmisartan- | % of Simvastatin- |
|---|---|---|---|
| Constituents | tablet | layer | layer |
| Telmisartan | 40.000 | 16.667 | |
| Sodium hydroxide | 3.360 | 1.400 | |
| Povidone | 12.000 | 5.000 | |
| Meglumine | 12.000 | 5.000 | |
| Sorbitol | 168.640 | 70.267 | |
| Magnesium stearate | 4.000 | 1.667 | |
| Purified water* | * | * | |
| Total Telmisartan-layer | 240.000 | 100.000 | |
| Simvastatin | 80.000 | | 40.000 |
| Microcrystalline | 40.000 | | 20.000 |
| cellulose | | | |
| Lactose monohydrate | 68.460 | | 34.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 440.000 | | |

*Volatile component, does not remain in final product

### Example 7

### Telmisartan 40 mg/Simvastatin 20 mg 2-Layer Tablets

| | mg per | % of Telmisartan- | % of Simvastatin- |
|---|---|---|---|
| Constituents | tablet | layer | layer |
| Telmisartan | 40.000 | 16.667 | |
| Sodium hydroxide | 3.360 | 1.400 | |
| Povidone | 12.000 | 5.000 | |
| Meglumine | 12.000 | 5.000 | |
| Sorbitol | 168.640 | 70.267 | |
| Magnesium stearate | 4.000 | 1.667 | |
| Purified water* | * | * | |
| Total Telmisartan-layer | 240.000 | 100.000 | |
| Simvastatin | 20.000 | | 10.000 |
| Microcrystalline | 40.000 | | 20.000 |
| cellulose | | | |
| Lactose monohydrate | 128.460 | | 64.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 440.000 | | |

*Volatile component, does not remain in final product

### Example 8

### Telmisartan 40 mg/Simvastatin 10 mg 2-Layer Tablets

| | mg per | % of Telmisartan- | % of Simvastatin- |
|---|---|---|---|
| Constituents | tablet | layer | layer |
| Telmisartan | 40.000 | 16.667 | |
| Sodium hydroxide | 3.360 | 1.400 | |
| Povidone | 12.000 | 5.000 | |
| Meglumine | 12.000 | 5.000 | |
| Sorbitol | 168.640 | 70.267 | |
| Magnesium stearate | 4.000 | 1.667 | |
| Purified water* | * | * | |
| Total Telmisartan-layer | 240.000 | 100.000 | |
| Simvastatin | 10.000 | | 5.000 |
| Microcrystalline | 40.000 | | 20.000 |
| cellulose | | | |
| Lactose monohydrate | 138.460 | | 69.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 440.000 | | |

*Volatile component, does not remain in final product

### Example 9

### Telmisartan 80 mg/Simvastatin 40 mg 2-Layer Tablets

| | mg per | % of Telmisartan- | % of Simvastatin- |
|---|---|---|---|
| Constituents | tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Sorbitol | 337.280 | 70.267 | |
| Magnesium stearate | 8.000 | 1.667 | |
| Purified water* | * | * | |
| Total Telmisartan-layer | 480.000 | 100.000 | |
| Simvastatin | 40.000 | | 20.000 |
| Microcrystalline | 40.000 | | 20.000 |
| cellulose | | | |
| Lactose monohydrate | 108.460 | | 54.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 680.000 | | |

*Volatile component, does not remain in final product

### Example 10

### Telmisartan 80 mg/Simvastatin 10 mg 2-Layer Tablets

| | mg per | % of Telmisartan- | % of Simvastatin- |
|---|---|---|---|
| Constituents | tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Sorbitol | 337.280 | 70.267 | |
| Magnesium stearate | 8.000 | 1.667 | |
| Purified water* | * | * | |
| Total Telmisartan-layer | 480.000 | 100.000 | |
| Simvastatin | 10.000 | | 5.000 |
| Microcrystalline | 40.000 | | 20.000 |
| cellulose | | | |
| Lactose monohydrate | 138.460 | | 69.230 |
| Hydroxypropyl | 4.000 | | 2.000 |
| methylcellulose | | | |
| Sodium starch glycolate | 6.000 | | 3.000 |
| Magnesium stearate | 1.500 | | 0.750 |
| Butylated hydroxyanisole | 0.040 | | 0.020 |
| Purified water* | * | | * |
| Ethanol* | * | | * |
| Total Simvastatin-layer | 200.000 | | 100.000 |
| Total 2-layer tablet | 680.000 | | |

*Volatile component, does not remain in final product

We claim:

1. A pharmaceutical tablet comprising:
(a) a first layer of telmisartan in a dissolving tablet matrix; and
(b) a second layer of simvastatin in a disintegrating or eroding tablet matrix.

2. The tablet according to claim 1, wherein telmisartan is in a substantially amorphous form.

3. The tablet according to claim 1, wherein the dissolving tablet matrix has instant release characteristics.

4. The tablet according to claim 1, wherein the dissolving tablet matrix comprises a basic agent and a water-soluble diluent.

5. The tablet according to claim 4, wherein the dissolving tablet matrix further comprises other excipients and adjuvants.

6. The tablet according to claim 5, wherein the other excipients and adjuvants are binders, carriers, fillers, lubricants, flow control agents, crystallization retarders, solubilizers, coloring agents, pH control agents, surfactants, and/or emulsifiers.

7. The tablet according to claim 4, wherein the basic agent is an alkali metal hydroxide, a basic amino acid, or meglumine.

8. The tablet according to claim 4, wherein the water-soluble diluent is a monosaccharide, a oligosaccharide; or a sugar alcohol.

9. The tablet according to claim 8, wherein the water-soluble diluent is glucose, sucrose, lactose, sorbitol, mannitol, or xylitol.

10. The tablet according to claim 1, wherein the first layer of telmisartan is produced by spray-drying an aqueous solution comprising telmisartan and a basic agent to obtain a spray-dried granulate, mixing the spray-dried granulate with a water-soluble diluent to obtain a premix, mixing the premix with a lubricant to obtain a final blend, and compressing the final blend to form the first tablet layer.

11. The tablet according to claim 1, wherein the disintegrating or eroding tablet matrix of the second layer comprises a filler, a lubricant, and an antioxidant.

12. The tablet according to claim 11, wherein the disintegrating or eroding tablet matrix of the second layer further comprises a binder.

13. The tablet according to claim 11, wherein the disintegrating or eroding tablet matrix of the second layer further comprises a disintegrant.

14. The tablet according to claim 11, wherein the disintegrating or eroding tablet matrix of the second layer further comprises other excipients and adjuvants.

15. The tablet according to claim 14, wherein the other excipients and adjuvants are chelating agents and/or coloring agents.

16. The tablet according to claim 1, wherein the first layer contains 10 mg to 160 mg of telmisartan.

17. The tablet according to claim 16, wherein the first layer contains 20 mg to 80 mg of telmisartan.

18. The tablet according to claim 17, wherein the first layer contains 40 mg to 80 mg of telmisartan.

19. The tablet according to claim 1, wherein the second layer contains 1 mg to 100 mg, of simvastatin.

20. The tablet according to claim 19, wherein the second layer contains 5 mg to 80 mg of simvastatin.

21. Tablets according to claim 1 packaged in a moisture proof packaging material.

22. Tablets according to claim 1 packaged in an aluminum foil blister pack, polypropylene tube, or an HDPE bottles.
